(19) United States
(12) Patent Application Publication
(10) Pub. No.: US 2018/0361314 A1
(43) Pub. Date: Dec. 20, 2018

(54) PROCESS AND APPARATUS FOR MANUFACTURING CARBON MONOXIDE

(71) Applicants: Lars-Erik Gärtner, Dresden (DE); Krish Krishnamurthy, Piscataway, NJ (US); Devin Bostick, Chester, NJ (US); Andreas Rupieper, Dresden (DE)

(72) Inventors: Lars-Erik Gärtner, Dresden (DE); Krish Krishnamurthy, Piscataway, NJ (US); Devin Bostick, Chester, NJ (US); Andreas Rupieper, Dresden (DE)

(21) Appl. No.: 15/624,756

(22) Filed: Jun. 16, 2017

(51) Int. Cl. B01D 53/32; C25B 3/04; C25B 15/08; G01N 33/00

(52) U.S. Cl. CPC B01D 53/326; C25B 3/04; B01D 2257/504; G01N 33/004; B01D 2256/20; C25B 15/08

(57) ABSTRACT

A process and apparatus for providing a carbon monoxide rich gas product. Carbon dioxide gas is partially converted in an electrolyser to obtain carbon monoxide gas and oxygen gas. A gas mixture containing at least carbon dioxide, carbon monoxide and hydrogen is withdrawn from the electrolyser. At least a part of the gas mixture is introduced into a separation system to provide a first gas or gas mixture enriched in carbon dioxide and a second gas or gas mixture enriched in carbon monoxide. The first gas or gas mixture or a part thereof is reintroduced into the electrolyser which is operated at a conversion rate for carbon dioxide 20% to 40% below a maximum conversion rate for the electrolyser.

The present invention relates to a process and an apparatus for manufacturing carbon. monoxide.

### PRIOR ART

For consumers which require carbon monoxide in an amount too small for standard steam methane reforming (SMR) to be cost-effective, carbon monoxide can be provided via the electrochemical reduction of carbon dioxide in an advanced electrochemical cell, as e.g. disclosed in US 2011/0157174 A1, US 2014/0093799 A1, US 2014/0239231 A1 and EP 0 489 555 B1. Electrochemical cells suitable for and/or adapted to the electrochemical reduction of carbon dioxide are, in the language as used herein, referred to as “electrolysers”. An electrolyser can comprise one or more electrochemical cells. As to further details, reference is made to expert literature and the patent documents cited above.

The carbon dioxide fed to a corresponding electrolyser can be captured from environmentally harmful carbon rich sources such as coal fired power plant flue gas using well tested, commercially-ready processes as generally known from the prior art. Therefore, providing carbon monoxide from carbon dioxide by electrochemical reduction is an environmentally friendly process.

The object of the present invention is to increase the general effectiveness of such carbon dioxide electrolysis processes so that carbon dioxide can be converted to carbon monoxide at a commercial scale, e.g. for use as a feedstock in applications such as electronics and specialty chemicals or fuels manufacturing.

### DISCLOSURE OF THE PRESENT INVENTION

This object is solved by a method and an apparatus for producing carbon monoxide as described herein.

At a first glance, each apparatus used in a process like the present should be operated at its best performance possible. In the present case, it would therefore generally be considered advantageous to operate the electrolyser under conditions optimised to obtain a maximum carbon monoxide product amount. Such an operation would also maximise the carbon dioxide conversion, providing a supposed further advantage. A corresponding operation could be used to establish a prima facie very simple process layout in the form of a “once through” method wherein the carbon monoxide product of the electrolyser would be used as such and the comparatively low amount of carbon dioxide resulting from the optimised conditions would be simply disposed of or used in other contexts. One could also contemplate, if this is acceptable for a specific application, to use a corresponding gas mixture with a low carbon dioxide content as a product without any further separation.

However, in the context of the present invention, it was found particularly advantageous to actually reduce the conversion rate of carbon dioxide in the electrolyser and therefore the amount of carbon monoxide product produced “per pass,” i.e. when directly comparing a composition of a gas or gas mixture immediately upstream of, and introduced into, the electrolyser to a composition of a gas or gas mixture immediately downstream of, and withdrawn from, the electrolyser. The reason for that is that the specific energy consumption was recognized to be significantly and disproportionately higher under the maximum conversion conditions. The present invention therefore proposes to increase the overall cost and energy effectiveness of the carbon monoxide production by operating of the electrolyser at a lower conversion per pass. This is possible in the context of the invention without losing valuable (and probably having to dispose of environmentally harmful) carbon dioxide by incorporating a downstream separation to separate unreacted carbon dioxide and recycling it back to the electrolyser. The use of such a recycle enables a higher overall conversion of the carbon dioxide in the electrolyser in spite of the reduced conversion per pass.

In the language as used herein, the terms “pressure level” and “temperature level” were chosen to characterise pressures and temperatures, this being intended to express that such pressures and temperatures do not have to be exact pressure or temperature values. Pressures and temperatures of pressure and temperature levels may vary within certain ranges, e.g. ±1%, 5%, 10%, 20% or even 50% about a mean or an average value. Pressure levels and temperature levels can lie in disjoint ranges or in ranges which overlap one another. In particular, pressure levels also cover different pressures which result from inevitable pressure drops or pressure losses. This correspondingly also applies to temperature levels. Pressure levels indicated herein in bar values are absolute pressures.

In the language as used herein, furthermore, liquid and gaseous mixtures are denoted as being rich or poor in one or more components, wherein “rich” can stand for a content of at least 50%, 75%, 90%, 95%, 99%, 99.5%, 99.9% or 99.99%, and “poor” can stand for a maximum content of 50%, 25%, 10%, 5%, 1%, 0.1% or 0.01% on a molar, weight or volume basis. If a mixture is characterized herein as comprising “predominantly” one or more components, the content in the one or more components may correspond to that as defined for the term “rich”. Liquid and gaseous mixtures may be described as being “enriched” or “depleted” in one or more components, these terrns relating to a corresponding content in of the one or more components in a starting mixture from which the liquid or gaseous mixture was obtained. The liquid or gaseous mixture is enriched when it contains at least the 1.1-fold, 1.5-fold, 2-fold, 5-fold, 10-fold, 100-fold or 1000-fold content, and it is depleted when it contains at most the 0.9-fold, 0.5-fold, 0.1-fold, 0.01-fold or 0.001-fold content of a corresponding component, with reference to the starting mixture. In the present case, e.g. when reference is made to “carbon monoxide” or “carbon dioxide” or respectively to a “carbon monoxide product”, what should also be understood by this is a mixture which is rich in the corresponding component. However, this may also refer to the respective pure gas.

According to the present invention, a process for providing a carbon monoxide rich gas product is provided. In the process of the present invention, carbon dioxide gas is partially converted in an electrolyser by electrolytic reduction to carbon monoxide gas and oxygen gas and from the electrolyser a gas mixture containing at least carbon dioxide gas, carbon monoxide gas and hydrogen gas is withdrawn. According to the present invention, at least a part of the gas mixture withdrawn from the electrolyser is introduced into a separation system providing a first gas or gas mixture which is, in the sense of the definitions above, enriched M carbon dioxide when compared to the gas mixture withdrawn from the electrolyser, and a second gas or gas mixture which is, in the sense of the definitions above, enriched in carbon monoxide when compared to the gas mixture withdrawn from the electrolyser. The first gas or gas mixture or a part thereof is reintroduced into the electrolyser. The second gas or gas mixture or a part thereof may be provided as the carbon monoxide rich gas product of the process. The first gas or gas mixture may, in the sense of the definitions above, also be rich in carbon dioxide and the second gas or gas mixture may, in the sense of the definitions above, also be rich in carbon monoxide.

According to the present invention, the electrolyser is operated at a conversion rate for carbon dioxide below, particularly 20% to 40% below, e.g. at about 30% below, a maximum conversion rate the electrolyser is practically able to achieve. The actual value used for conversion in the electrolyser can be determined in form of a “sweet spot” at which the process can be operated at a minimum energy consumption at a maximum overall (i.e. not per instance or per pass through the electrolyser, as explained above) carbon monoxide production. In the context of the present invention, the “maximum conversion rate the electrolyser is practically able to achieve” can be e.g. determined by pre-experiments or deduced from technical or manufacturer data without undue burden to the practitioner.

The advantages achieved by the present invention therefore particularly include a reduction in power consumption in the electrolyser by operating it at a lower once-through conversion of carbon dioxide but still maintaining or increasing the overall conversion by recycling carbon dioxide from the downstream separation system, the first gas or gas mixture or a part thereof, back to the electrolyser, as already explained above in more detail.

According to a particularly preferred embodiment of the present invention, the electrolyser is operated at a pressure level, in the following referred to as “electrolysing pressure level,” above atmospheric pressure, e.g. at a pressure level of at least 3, 4 or 5 bar. Particularly, the electrolysing pressure level may be in a range of 3, 4 or 5 bar to 30 bar, especially in a range of 10 to 30 bar, preferably in a range of 20 to 30 bar. Using an elevated electrolysing pressure level particularly allows for introducing the gas mixture withdrawn from the electrolyser or the part thereof into the separation system without, or at least without any significant, compression upstream of the separation system. The separation system is typically operated at, at least an initial, pressure level, in the following referred to as “separating pressure level,” at or around the electrolysing pressure level. In other words, the gas mixture withdrawn from the electrolyser or the part thereof is submitted to the separation system at the separating pressure level. This allows for an additional reduction of capital and operating costs as a corresponding compressor and its operation can be dispensed of.

The reduction in capital and operating costs for the integrated carbon dioxide electrolyser and downstream carbon monoxide purification according to this embodiment of the present invention is, in other words a result of the elimination of a substantial intermediate compression step upstream of the gas purification process that would otherwise be required for an electrolyser operating at low or ambient pressure. The main disadvantages of the prior art that can be eliminated according to this embodiment of the present invention is therefore particularly the requirement for an energy intensive and expensive compression step upstream of the carbon monoxide purification process when carbon dioxide electrolysis is carried out at low or ambient pressure. However, in other embodiments of the present invention, the electrolyser can also be operated at a lower electrolysing pressure level, particularly at or around atmospheric pressure.

According to the previously discussed embodiment of the present invention, the electrolyser may be operated at an electrolysing pressure level differing by no more than 1 bar, particularly by no more than 0.5 bar or by no more than 0.1 bar from the separating pressure level. In other words, and as already mentioned, the electrolysing step may be performed at the elevated pressure level used in the at least one separation system. This alternative is particularly preferable because no intermediate compressor whatsoever is necessary in between the electrolysing step and the separation system. The prerequisite to successfully perform the method of the present invention according to this alternative is, however, that the electrolysing step may viably be performed at corresponding elevated pressure level.

According to a further alternative of the present invention, which may be particularly used when the electrolysing step may not viably be performed at a corresponding elevated pressure level, the electrolyser may be operated at an electrolysing pressure level of 1.5 to 2.5 bar, especially of 1.75 to 2.25 bar, particularly of 1.9 to 2.1 bar, for example at atmospheric pressure. This alternative may be advantageous because the electrolyser does not need to be built to withstand elevated pressures.

In this alternative, e.g. if the electrolyser is operated at an electrolysing pressure level. of 1.5 to 2.5 bar or the sub-ranges mentioned above, the gas mixture withdrawn from the electrolyser or the part thereof submitted to the separation system is preferably compressed in a gas compressor from the electrolysing pressure level to the separating pressure level. Even if, in this alternative of the present invention, a gas compressor is used, the inventive method is still advantageous because the pressure increase that needs to be generated by a corresponding gas compressor is substantially lower than in methods of the prior art.

According to preferred embodiments of the present invention, the separation system may be adapted to perform a pressure swing adsorption or a membrane-based separation, as generally known from the prior art. As also mentioned with regard to FIGS. 1A and 1B, the separation system may be adapted to perform one separation step, in which case a product stream thereof may still contain noticeable amounts of hydrogen and carbon dioxide gas from the carbon dioxide feed stream or generated in the electrolyser from water. However, if a corresponding hydrogen or carbon dioxide content is acceptable, this alternative enables for a particularly cost-effective setup.

In contrast, if such a hydrogen or carbon dioxide content is not acceptable, a further alternative of the present invention may be more preferable. In this further alternative, which is also described with reference to FIGS. 2A and 2B, more than one separation step is performed, wherein at least a portion of the gas mixture withdrawn from the electrolyser is submitted to one of the separation steps. The second gas or gas mixture enriched in carbon monoxide is then withdrawn from another one of the more than one separation steps. Increasing the number of separation steps, an ever-increasing purity of the second gas or gas mixture enriched in carbon monoxide can be obtained. In this case, at least a portion of the gas mixture withdrawn from the electrolyser is submitted to a “first” of a chain of such separation steps and the second gas or gas mixture enriched in carbon monoxide is withdrawn from a “last” separation step in a chain.

Generally, the process of the present invention, the carbon dioxide gas is provided to the electrolyser at a temperature level from 35 to 100° C., particularly at a temperature level from 35 to 80° C., for example at a temperature level from 35 to 50° C., from 50 to 65° C., from 65 to 80° C. or from 80 to 100° C. The specific temperature level used particularly depends on the electrolyser at different temperatures.

The carbon dioxide gas may be provided to the electrolyser with a purity of at least 95.0 mol % on a dry basis, particularly with a purity of at least 99.0 mol % on a dry basis, especially with a purity of at least 99.999 mol % on a dry basis. The carbon dioxide gas with a corresponding specification may be obtained using the processes as mentioned in the outset.

According to a particularly preferred embodiment of the present invention, the carbon dioxide gas is provided to the electrolyser at the electrolysing pressure level which eliminates the need for compression and/or expansion means.

The present invention also relates to an apparatus for providing a carbon monoxide rich gas product, the apparatus including an electrolyser adapted to partially convert carbon dioxide gas by electrolytic reduction to carbon monoxide gas and oxygen gas and gas mixture containing carbon dioxide gas, carbon monoxide gas and hydrogen gas from the electrolyser. According to the present invention, such an apparatus comprises a separation system and means that are adapted to submit at least a part of the gas mixture withdrawn from the electrolyser to the separation system. According to the present invention, the separation system is adapted to provide a first gas or gas mixture enriched in carbon dioxide when compared to the gas mixture withdrawn from the electrolyser and a second gas or gas mixture enriched in carbon monoxide when compared to the gas mixture withdrawn from the electrolyser, and means are provided to reintroduce the first gas or gas mixture or a part thereof into the electrolyser. Furthermore, means are provided adapted to operate the electrolyser is at a conversion rate for carbon dioxide which is 20% to 40% below a maximum conversion rate the electrolyser is practically able to achieve

As to features and advantages of the apparatus provided according to the present invention, reference is made to the explanations relating to the inventive method above. Particularly, as the apparatus may include means adapted to perform a method as described above.

The present invention is further exemplified on the basis of the appended drawings showing preferred embodiments of the present invention.

### SHORT DESCRIPTION OF THE FIGURES

FIG. 1A illustrates a process according to a preferred embodiment of the present invention.

FIG. 1B illustrates an alternative to the process illustrated in FIG. 1A according to a further preferred embodiment of the present invention.

FIG. 2A illustrates a process according to a further preferred embodiment of the present invention.

FIG. 2B illustrates an alternative to the process illustrated in FIG. 2A according to a further preferred embodiment of the present invention.

FIG. 3A illustrates a process according to a further preferred embodiment of the present invention.

FIG. 3B illustrates an alternative to the process illustrated in FIG. 3A according to a further preferred embodiment of the present invention.

In the figures, like and/or functionally corresponding elements are identified with identical reference numerals. For reasons of conciseness, repeated explanations of such elements are omitted.

### DETAILED DESCRIPTION OF THE FIGURES

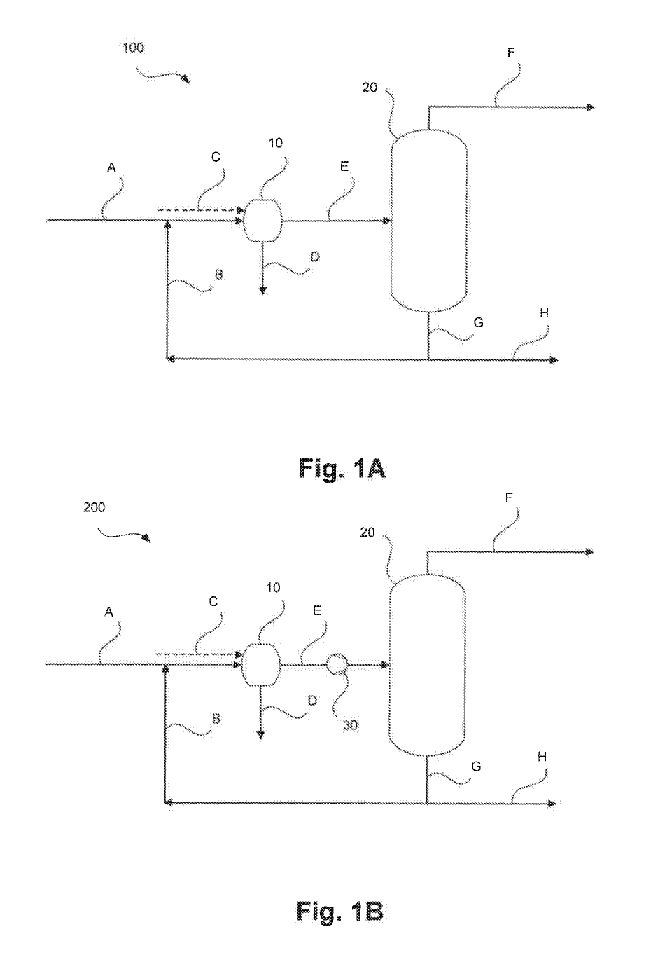

In FIG. 1A, a process according to a preferred embodiment of the present invention is schematically illustrated and designated 100.

According to the process 100 as shown in FIG. 1A, a carbon dioxide rich gas stream A, preferably containing at least 99.9 mol % carbon dioxide on a dry basis, is fed together with an optional recycle stream B described below at a pressure level of at least 5 bar and at a temperature level of 35 to 100° C. into a carbon dioxide electrolyser 10, wherein, under application of electricity C illustrated as a dashed arrow, carbon dioxide is electrochemically reduced forming carbon monoxide and oxygen. The electrolyser 10 is preferably embodied as a stack of electrolyser cells and operates at the pressure level (also referred to as an “electrolysing pressure level” herein) at which the carbon dioxide rich gas stream A is provided, i.e. at least 5 bar. Oxygen gas leaves an anode of the electrolyser 10 as an oxygen rich gas stream D. A carbon monoxide enriched gas stream E still containing residual hydrogen from the carbon dioxide rich gas stream A and carbon dioxide not converted in the electrolyser 10 is supplied to a separation system 20. In the process 100 as shown in FIG. 1A, the separation system 20 also operates at a pressure level (also referred to as a “separation pressure level” herein) of at least 5 bar. From the separation system 20, a carbon monoxide rich gas stream F (previously referred to as a “second” gas or gas mixture) and a further gas stream G mostly containing the separated carbon dioxide and hydrogen (previously referred to as a “first” gas or gas mixture) are withdrawn. The further gas stream G is in part be recycled in form of the recycle stream B to the electrolyser 10 in order to increase the carbon dioxide conversion. As mentioned, the electrolyser 10 is particularly operated at a conversion rate below a practical maximum.

As, according to the process 100 as shown in FIG. 1A, the carbon monoxide rich gas stream F still contains noticeable amounts of hydrogen, this process is preferred if a corresponding content in the carbon monoxide product corresponding to the carbon monoxide rich gas stream F is desired or can be tolerated.

In FIG. 1B, an alternative to the process illustrated in FIG. 1A according to a preferred embodiment of the present invention is schematically illustrated and designated 200.

In contrast to process 100 shown in FIG. 1A, in the process 200 as shown in FIG. 1B the carbon dioxide rich gas stream A is provided at a pressure level of only about I bar. The temperature level and the carbon dioxide content may be comparable to that explained for the process 100 as shown in FIG. 1A. The electrolyser 10 correspondingly operates at a lower electrolysing pressure level which corresponds to the lower pressure level of the carbon dioxide rich gas stream A, i.e. at about 1 bar. The carbon monoxide enriched gas stream E is compressed in a gas compressor 30 before being provided to the separation system 20, which operates at the same separation pressure level at as the separation system 20 according to the process 100 as shown in FIG. 1A, i.e. at a pressure level of at least 5 bar. The carbon monoxide enriched gas stream E leaves the gas compressor 30 at this pressure level, i.e. at least 5 bar.

Like the process 100 as shown in FIG. 1A, the carbon monoxide rich gas stream F of the process 200 according to FIG. 1B still contains noticeable amounts of hydrogen. The process 200 is therefore also preferred if a corresponding content in the carbon monoxide product is desired or can be tolerated. Furthermore, the process 200 according to FIG. 1B is preferably used if the electrolysis cannot be viably achieved at the electrolysing pressure used according to process 100.

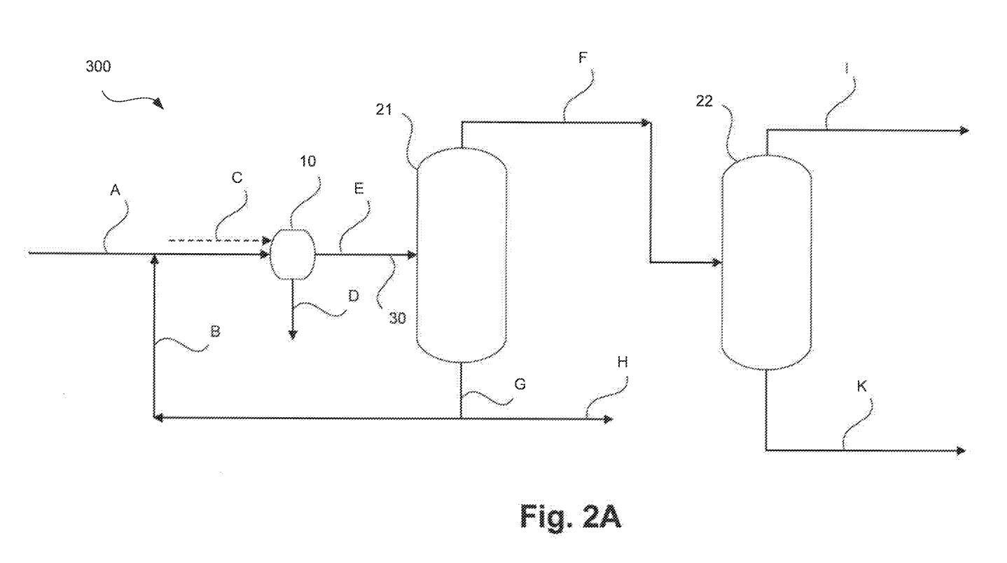

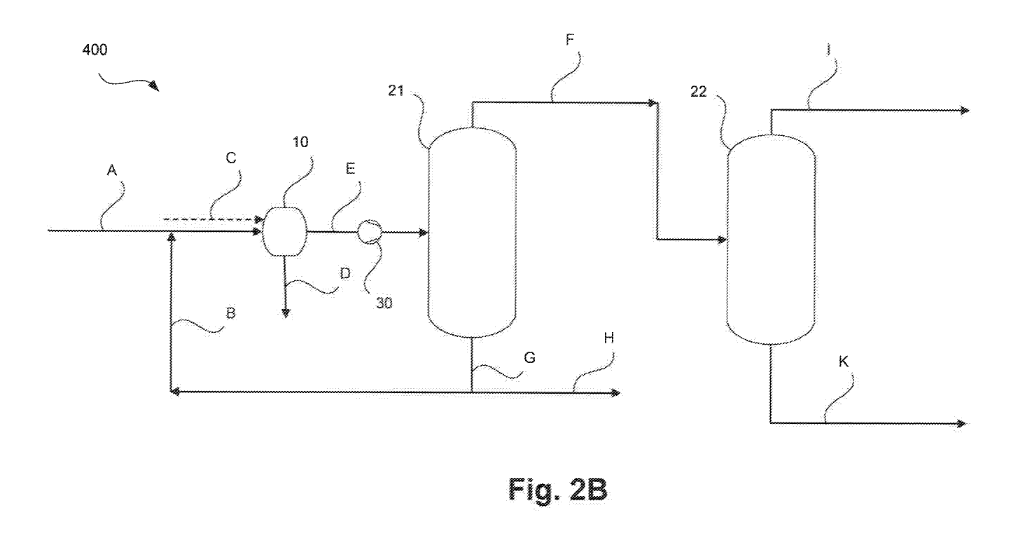

In FIG. 2A, a one-stage process according to a further preferred embodiment of the present invention is schematically illustrated and designated 300.

According to the process 300 as shown in FIG. 2A, the carbon dioxide rich gas stream A is provided under the conditions used in the process 100 as shown in FIG. 1A, i.e. at a pressure level of at least 5 bar, a temperature level of 35 to 100° C. and with a carbon dioxide content of preferably at least 99.99 mol % carbon dioxide on a dry basis. The electrolyser 10 used according to the process 300 shown in FIG. 2A operates at the electrolysing pressure level which corresponds to the pressure level at which the carbon dioxide rich gas stream A is provided, i.e. at a pressure level of at least 5 bar. In contrast to the process 100 as shown in FIG. 1, however a two-stage separation system with steps or units 21 and 22 is performed. Both steps or units 21 and 22 operate at a separation pressure level which corresponds to the electrolysing pressure level at which the electrolyser 10 operates and at which the carbon dioxide rich gas stream A is provided, i.e. at a pressure level of least 5 bar. The process 300 according to FIG. 2A essentially differs from the process 100 according to FIG. 1A in that the carbon monoxide rich gas stream F is not directly provided as a product but is further purified in the (second) step or unit 22. This carbon monoxide rich gas stream F which, as mentioned, still contains noticeable amounts of hydrogen, is separated in the second step or unit 22 into a hydrogen product gas withdrawn from the second step or unit 22 as a product stream I and into a further purified carbon monoxide product gas withdrawn from the second step or unit 22 as a product stream K.

As according to the process 300 as shown in FIG. 2A, a further step or unit 22 is used, the carbon monoxide product gas withdrawn as the product stream K is further depleted in hydrogen as compared to the carbon monoxide rich gas stream F. Process 300 as shown in FIG. 2A is thus preferred if higher purity levels of the carbon monoxide product are desired or necessary.

In FIG. 2B, an alternative to the process illustrated in FIG. 2A according to a further preferred embodiment of the present invention is schematically illustrated and designated 400.

In contrast to process 300 as shown in FIG. 2A, in the process 400 as shown in FIG. 2B the carbon dioxide rich gas stream A is provided at a pressure level of only about 1 bar. Therefore, the process 400 as shown in FIG. 2B partially resembles the process 200 as shown in FIG. 1B. The temperature level and the carbon dioxide content of the carbon dioxide rich gas stream A may be comparable to that used in the processes 100, 200 and 300 as discussed before. The electrolyser 10 correspondingly operates at the lower electrolysing pressure level of the carbon dioxide rich gas stream A, i.e. at about 1 bar. The carbon monoxide enriched gas stream E is compressed in a gas compressor 30 before being provided to the (first) step or unit 21 which operates at the same separation pressure level as the separation system 20 according to the processes 100, 200 and the steps or units 21 and 21 of the process 300 as discussed before, i.e. at a separation pressure level of at least 5 bar. The carbon monoxide enriched gas stream E leaves the gas compressor 30 at this pressure level, i.e. at least 5 bar. For further explanations, reference is made to the explanations to FIG. 2A.

As according to the process 400 as shown in FIG. 2B, like in the process 300 as shown in FIG. 2A, a further step or unit 22 is provided, the carbon monoxide product gas withdrawn from the activated carbon PSA as the gas stream K is further depleted in hydrogen as compared to the carbon monoxide rich gas stream F. The process 400 as shown in FIG. 2B is thus preferred if higher purity levels of the product streams are desired or necessary. Furthermore, in the process 400 as shown in FIG. 2B, the electrolyser 10 operates at a lower electrolysing pressure level than the electrolyser 10 according to the process 300 as shown in FIG. 2A. Therefore, process 400 according to FIG. 2B is desirable if the electrolysis cannot be viably achieved at the pressure level used according to process 300.

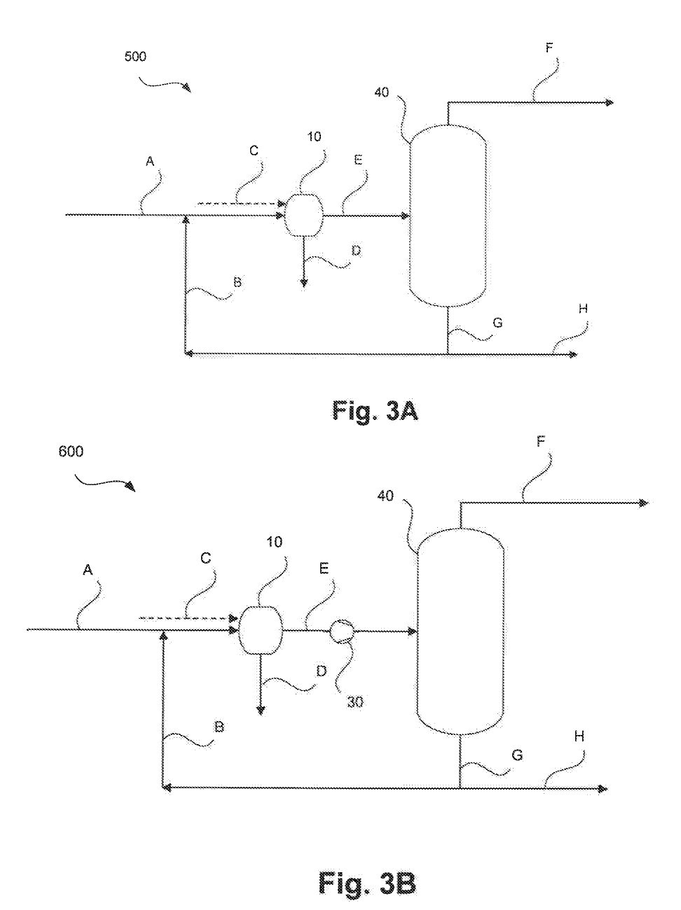

In FIG. 3A, a membrane-based gas purification process according to a further preferred embodiment of the present invention is schematically illustrated and designated 500.

The pressure level and further specifications of the carbon dioxide rich gas stream. A correspond to that of the carbon dioxide rich gas stream A used in the processes 100 and 300 as shown in FIGS. 1A and 2A. These conditions are not repeated for the sake of conciseness. Also the electrolysing pressure level of the carbon dioxide electrolyser 10 in the process 500 according FIG. 3A resembles that of the carbon dioxide electrolysers 10 in the processes 100 and 300 as shown in FIGS. 1A and 2A. In contrast to the processes 100, 200, 300 and 400 as shown in FIGS. 1A, 1B, 2A and 2B, however, a different separation system 40 is used. Depending on the operating performance of the separation system 40, the specifications of the streams F, G and H may resemble that of the corresponding streams F, G and H of the processes 100, 200, 300 and 400 as discussed before or may differ.

In FIG. 3B, an alternative to the process illustrated in FIG. 3A according to a further preferred embodiment of the present invention is schematically illustrated and designated 600.

The essential difference between the process 500 illustrated in FIG. 3A and the process 600 illustrated in FIG. 3B is that the carbon dioxide rich gas stream A is provided at a pressure level which is comparable to that used in the processes 1B and 2B, i.e. at about 1 bar. The electrolysing pressure level at which the electrolyser 10 is operated, is also about 1 bar. As the different separation system 40, however, operates at a separating pressure level comparable to that used in the process 500 according FIG. 3A, like in the processes 200 and 400 illustrated in FIGS. 1B and 2B a gas compressor 30 is used.

It is understood that the separation system used may incorporate a pressurizing device such as a blower or a compressor to overcome the pressure drops through the system and return the CO2 rich stream for recycle to the electrolyzer.

1. A process for providing a carbon monoxide rich gas product, wherein carbon dioxide gas is partially converted in an electrolyser by electrolytic reduction to carbon monoxide gas and oxygen gas and wherein a gas mixture containing at least carbon dioxide gas, carbon monoxide gas and hydrogen gas is withdrawn from the electrolyser,
characterised in that
at least a part of the gas mixture withdrawn from the electrolyser is introduced into a separation system providing a first gas or gas mixture enriched in carbon dioxide when compared to the gas mixture withdrawn from the electrolyser and a second gas or gas mixture enriched in carbon monoxide when compared to the gas mixture withdrawn from the electrolyser, in that the first gas or gas mixture or a part thereof is reintroduced into the electrolyser, and in that the electrolyser is operated at a conversion rate for carbon dioxide which is 20% to 40% below a maximum conversion rate the electrolyser is practically able to achieve.

2. A process according to claim 1, wherein the electrolyser is operated at a electrolysing pressure level of at least 3 bar.

3. A process according to claim 2, wherein the gas mixture withdrawn from the electrolyser or the part thereof is submitted to the separation system at a separation pressure level of at least 3 bar.

4. A process according to claim 3, wherein the electrolysing pressure level is differing by no more than 1 bar from the separation pressure level.

5. A process according to claim 1, wherein the electrolyser is operated at an electrolysing pressure level of 0.5 to 1.5 bar.

6. A process according to claim 5, wherein the gas mixture withdrawn from the electrolyser or the part thereof submitted to the separation system is compressed in a gas compressor from the electrolysing pressure level to the separating pressure level.

7. A process according to claim 1, wherein exactly one separation step is performed in the separation system, and wherein the second gas or gas mixture is withdrawn from the exactly one separation step.

8. A process according to claim 1, wherein more than one separation step is performed, wherein at least a portion of the gas mixture withdrawn from the electrolyser is submitted to one of the more than one separation steps, and wherein the second gas or gas mixture is withdrawn from another one of the more than one separation steps.

9. A process according to claim 1, wherein the carbon dioxide gas is provided to the electrolyser at a temperature level from 35 to 100° C.

10. A process according to claim 1, wherein the carbon dioxide gas is provided to the electrolyser with a purity of at least 99.9 mol % on a dry basis.

11. A process according to claim 2, wherein the carbon dioxide gas is provided to the electrolyser at the electrolysing pressure level.

12. An apparatus for providing a carbon monoxide rich gas product, including an electrolyser adapted to partially convert carbon dioxide gas by electrolytic reduction to carbon monoxide gas and oxygen gas and means to withdraw a gas mixture containing carbon dioxide gas, carbon monoxide gas and hydrogen gas from the electrolyser,
characterised in that
the apparatus comprises a separation system and means adapted to submit at least a part of the gas mixture withdrawn from the electrolyser to the separation system, the separation system being adapted to provide a first gas or gas mixture enriched in carbon dioxide when compared to the gas mixture withdrawn from the electrolyser and a second gas or gas mixture enriched in carbon monoxide when compared to the gas mixture withdrawn from the electrolyser, in that means are provided to reintroduce the first gas or gas mixture or a part thereof into the electrolyser, and in that means are provided that are adapted to operate the electrolyser at a conversion rate for carbon dioxide which is 20% to 40% below a maximum conversion rate the electrolyser is practically able to achieve.
